(19) United States
(12) Patent Application Publication
(10) Pub. No.: US 2009/0131919 A1
(43) Pub. Date: May 21, 2009

(54) Implantable medical device

(76) Inventor: Christopher Davey, Dublin (IE)

Correspondence Address: Kevin M. Farrell;Pierce Atwood, One New Hampshire Avenue, Suite 350, Portsmouth, NH, 03801, US

(21) Appl. No.: 11/986,451

(22) Filed: Nov. 21, 2007

(51) Int. Cl. A61K 9/22; A61M 5/32

(52) U.S. Cl. 604/891.1; 604/175

(57) ABSTRACT

The present invention comprises a modular, implantable medical device comprising a body portion capable of receiving a treatment device that accesses a patient's inner physiology while communicating with a device external to the patient. The body portion further includes a first skirt and a second skirt that surround the treatment device at its junction with the body portion. Both skirts are designed to gradually affix themselves into the surrounding tissues of the patient's body during an initial phase of healing. The first skirt is designed to separate from the device at a lower force than the second skirt such that during removal, the device and second skirt can be detached as a unit from patient's skin and first skirt without excessive force or trauma and the first skirt remains attached to the patient's skin as a permanent implant.

### BACKGROUND OF THE PRESENT INVENTION

1. Field of the Invention

The present invention relates generally to the field of medical devices and in particular to the field of long term, implantable devices for permitting access to a patient's inner physiology.

2. Summary of the Related Art

Medically treating a patient often requires long term placement of a medical device across different organ systems to establish access to a specifically targeted interior body site for diagnostic or therapeutic purposes. One common example is the establishment of percutaneous vascular access for purposes of administering liquid therapeutic agents, for removing bodily fluids for testing or monitoring, for treatment of bodily fluids before being returned to the body, or for disposal of bodily fluids.

Particularly in the case of administering fluids to, or removing fluids from, the body continuously or periodically over an extended time period, those skilled in the medical arts typically use what are known as “permanent” catheterization techniques employing implanted devices such as tunneled central venous catheters (CVCs) for durations ranging from a few weeks to years. Examples of such implanted and related medical devices exist in U.S. Pat. No. 4,266,999 (Baier); U.S. Pat. No. 4,405,305 (Stephen et al.); U.S. Pat. No. 4,488,877 (Klein et al.); U.S. Pat. No. 4,668,222 (Poirier); U.S. Pat. No. 4,897,081 (Poirier et al.); U.S. Pat. No. 4,935,004 (Cruz); U.S. Pat. No. 5,098,397 (Svensson et al.); U.S. Pat. No. 5,100,392 (Orth et al.); U.S. Pat. No. 5,156,597 (Verreet et al); U.S. Pat. No. 5,242,415 (Kantrowitz et al.); U.S. Pat. No. 5,662,616 (Bousquet); U.S. Pat. No. 5,823,994 (Sharkey et al.); U.S. Pat. No. 5,830,184 (Basta); U.S. Pat. No. 5,848,987 (Baudino et al.); U.S. Pat. No. 5,882,341 (Bousquet); U.S. Pat. No. 5,989,213 (Maginot); and U.S. Pat. No. 6,033,382 (Basta), each of which is incorporated herein by reference. Examples of therapeutic regimens requiring such long-term continuous or periodic access to a specific internal body location include parenteral feeding, chemotherapy, antibiotic administration, dialysis, chronic anesthesiology, and others.

Generally, the type of procedure that a patient requires dictates whether a physician will utilize an acute, short term catheterization technique, or a chronic, long term catheterization technique. For example, establishing a state of general anesthesiology in preparation for a surgical procedure typically involves placing a CVC in a patient's blood vessel for a relatively short period of time, such as a few minutes to a few hours, and then removing the catheter once the surgery is finished and the patient is revived. Thus, when performing such an anesthesiology procedure, physicians commonly use this short term catheterization technique to place a drug delivery catheter in a blood vessel of the patient.

In direct contrast to this example of short term CVC placement, a physician performing a hemodialysis procedure in a patient suffering from chronic kidney failure may place a CVC in one of the patient's blood vessels for a relatively long period of time. Such a patient typically requires dialysis sessions three times per week for an indefinite period of time. Healthy kidney function insures removal of fluid, chemicals, and wastes typically filtered from a person's blood. Hemodialiysis involves removing these elements by sending a patient's blood to an external artificial kidney machine via the permanent vascular access often established by placement of a long term catheter within the patient. A patient who is involved in such a hemodialysis regimen may need a catheter placed in a blood vessel for weeks, months, or years in order to provide a ready means for vascular access into that patient's bloodstream to enable these frequent life saving dialysis treatments.

Long term catheterization techniques typically entail inserting a catheter into a patient using a “tunneled catheter technique.” This procedure involves inserting a long term catheter into the patient through an incision in the skin and then routing the catheter for several centimeters under the skin before entering deeper regions of the body. Although used routinely, conventional tunneled catheter designs seriously compromise the ability of a patient's skin to protect the patient's body from infection. As discussed in “Intravascular Catheter-Related Infections: New Horizons and Recent Advances” (Raad et al., Arch Internal Medicine/Vol 162, Apr. 22, 2002, Pages 871-878), catheter-related infections are frequent events and present a potentially fatal health problem. High morbidity rate and subsequently high procedural cost therefore are characteristics of typical long term tunneled catheter usage. The primary reason that use of conventional catheters leads to a high rate of infection is that microorganisms enter the body through the skin incision. Although a conventional tunneled catheter may include a tissue in-growth cuff that acts as a barrier for micro-organisms entering the body and that anchors the device in the subcutaneous tunnel, such a conventional device still produces undesirably high infection rates because the standard design prevents positioning the cuff in the most effective location for preventing infection, the skin entry site.

Furthermore, in order to function properly over extended periods of time, many types of long term tunneled catheters require placement of their tips in a very specific high blood flow location, typically the Superior Vena Cava/Right Atrial Junction (SVC/RA). The turbulent flow in this location ensures rapid mixing and systemic distribution of therapeutic agents in the patient, and also minimizes the risk of thrombus forming on a catheter's tip and leading to catheter dysfunction. Skilled clinicians are acutely aware of the need for highly precise tip placement because they frequently diagnose and resolve catheter complications. With conventional tunneled catheter designs, the ability to precisely adjust the position of the catheter tip in the SVC/RA depends largely on a freedom to position and adjust the cuff anywhere along the length of a subcutaneous tunnel.

A tunneled catheter apparatus that includes an adjustable epidermal tissue ingrowth cuff assembly overcomes these problems and deficiencies of the prior art devices. The apparatus and methods disclosed in U.S. Patent Application No. 2004/0236314 to Mark A. Saab, incorporated herein by reference, allow a physician to place a fixed epidermal tissue ingrowth cuff assembly precisely within a skin incision site and subsequently adjust the location of the distal (internal) tip of a catheter assembly associated with the tissue ingrowth cuff assembly. A physician using such a device, therefore, can position the catheter tip precisely at the desired body site without disturbing, moving, or stressing the fixed tissue ingrowth cuff.

Nevertheless, while development of Saab's advanced tissue ingrowth cuff assemblies has resulted in numerous improvements related to patient care and health, a typical epidermal tissue ingrowth cuff has a significantly larger surface area than conventional cuffs. This increased surface area results in substantial tissue ingrowth that creates a high level of difficulty in detaching the device from a patient's physiology during device removal. A need therefore exists for providing an adequately large surface area cuff that promotes stable and secure attachment to living tissue during the tenuous early stages of the healing, but that avoids providing such an excessively robust degree of tissue ingrowth that excessive or undesirable levels of force and trauma are required to detach the device at the end of treatment or at a time for replacement. U.S. patent application Ser. No. 11/242,101 to Christopher Davey presents one novel means for addressing this deficiency. That application describes a cuff design at least partially comprising a bioabsorbable material. When initially placed, the Davey cuff has a large surface area to achieve a degree of stability that promotes healing. At the time of removal, the partially absorbed cuff has a significantly reduced surface area that allows for removal of the device and remaining cuff with a relatively reduced level of associated trauma. This device, however, requires proper timing such that the cuff can degrade sufficiently prior to removal of the device and remaining tissue ingrowth cuff material.

The present invention comprises a biocompatible cuff that provides stabilization and protection against infection, and further enables gentle, non-traumatic removal of an implanted cuff assembly at the end of the treatment process.

### SUMMARY OF THE PRESENT INVENTION

The present invention provides a medical device that is capable of implantation within a patient for long-term treatments, such as catheterization procedures. The device of the present invention includes an implantable body portion capable of receiving and anchoring a treatment device, such as a fluid conduit, power cable or fiber optic cable that extends through a living membrane, such as skin, and into a patient's internal physiology. The body portion is shaped to maximize comfort and ease of installation, and thus a relatively flat and generally rectangular geometry is most preferable for a variety of applications.

One embodiment of the device of the present invention comprises a modular design such that interior and exterior ports are selectively attachable to the body portion. The interior and exterior ports are connectable to the body portion through threading or other mechanical means known in the art. As with most medical devices, the interior and exterior ports as well as the body portion are crafted from a durable and sterilizable material, such as a thermoset polymer, stainless steel, or titanium.

In one embodiment, the device incorporates an assembly of tissue in-growth skirt materials that attach in concentric fashion to the top surface of the body portion. Skirt components readily may be made from an implant material such as Dacron®, a medical grade woven or knitted polyester material commonly used as a permanent implant during a wide range of surgical procedures. (For example, as discussed in “Dacron Implants in Rhinoplasty” (Fanous et al., Arch Facial Plastic Surgery/Vol 4, July-September 2002, Pages 149-156), tissue ingrowth scaffold materials of the type used for CVC cuff designs are used routinely in other clinical procedures as permanent implants.)

At least one of the skirt components bonds to a living membrane of the patient in such a manner as to enable separation from the body portion of the device with a predetermined and relatively low level of force as compared to other components. Upon initial assembly and placement of the device within the patient, the skirt assembly provides a large surface area that promotes tissue in-growth with the living membrane and securely anchors the device in position as the healing process begins and progresses. Once the healing process ends, the entire skirt assembly will have become highly interconnected with the surrounding tissues and could require a significant amount of force and trauma to effect complete removal of the device from the patient. By attaching a portion of the skirt in such a way that separation from the body portion of the device occurs at a low level of force, users of the present invention can remove the body portion of the device with little force and leave the detached portion of the skirt behind in the patient as a permanent implant.

These and other features and advantages of the device of the present invention are described in greater detail below with reference to the following figures.

### BRIEF DESCRIPTION OF THE DRAWINGS

FIG. 1 is a perspective view of an implantable medical device according to the present invention.

FIG. 2 is a cross-sectional view of the implantable medical device shown in FIG. 1.

FIG. 3 is an exploded perspective view of the implantable medical device shown in FIG. 1.

FIG. 4 is a plan view of the implantable medical device shown in FIG. 1.

FIG. 5 is a sectional view of the implantable medical device of the present invention shown implanted within a patient's body.

FIG. 6 is an exploded sectional view of the implantable medical device of the present invention shown implanted within a patient's body.

FIG. 7 is an exploded sectional view of the implantable medical device of the present invention shown during removal from a patient's body.

### DETAILED DESCRIPTION OF THE PREFERRED EMBODIMENTS

The present invention includes an implantable medical device 10 that is well adapted for providing long-term access to the inner physiology of a patient. One such application of this device is providing long-term vascular access for various kinds of catheterization and/or dialysis procedures. In particular, the present invention is readily usable as a tissue ingrowth cuff or similar device for enabling living membrane, such as skin, at the implant device entry site to heal into the device and block the path of pathogens that would otherwise infect the patient. A sufficiently large degree of tissue ingrowth therefore occurs during the initial phases of healing. Because the ingrowth cuff of the present invention includes detachable components, the device also enables uncomplicated, non-traumatic removal at the end of the healing process.

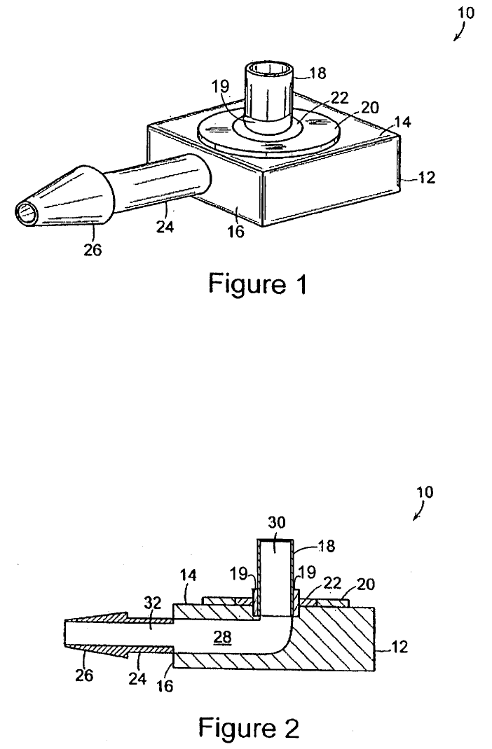

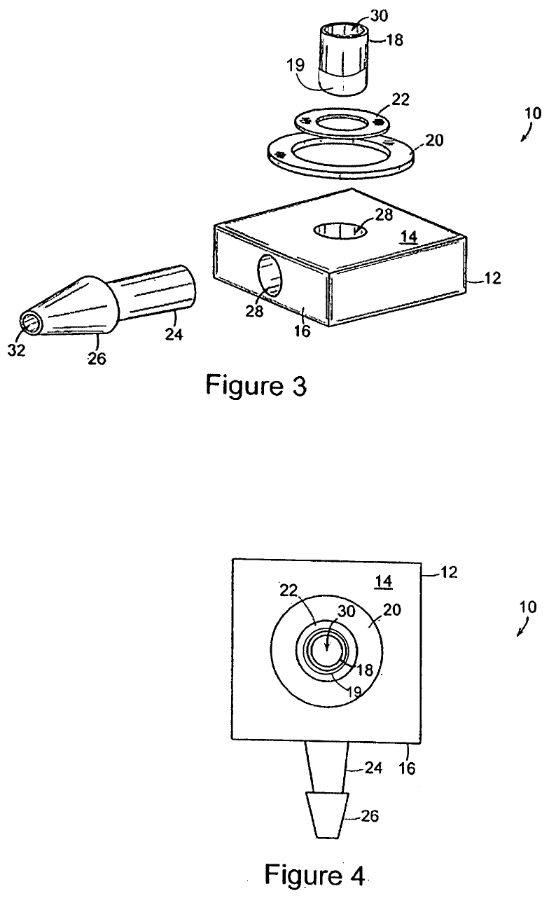

FIGS. 1 through 4 are various views of one embodiment of the device 10 of the present invention. In this embodiment, the device includes a body portion 12 that defines a first surface 14 and a second surface 16. In preferred embodiments, the first surface 14 and the second surface 16 are mutually orthogonal, and more preferably, the first surface 14 and the second surface 16 are arranged such that lines normal to each respective surface are mutually orthogonal. Although the body portion 12 shown in FIGS. 1-4 defines planar surfaces, equally suitable design choices are readily available to those skilled in the art.

In one embodiment, the body portion 12 is adapted to receive an exterior port 18 that defines an exterior lumen 30 for transporting fluids through the exterior port 18. The exterior port 18 is preferably cylindrical in shape, and the exterior lumen 30 preferably defines a cylindrical surface through the interior of the exterior port 18. Additionally, the exterior port 18 may incorporate a tissue ingrowth ring 19 disposed on the outer surface of the exterior port 18. In some embodiments, this tissue ingrowth ring 19 may extend along the entire length of the exterior port 18, but preferably, the tissue ingrowth ring 19 extends along the portion of the exterior port 18 that interfaces with the skin. Although a single lumen-type exterior port 18 is illustrated throughout FIGS. 1 through 7, alternative designs including multiple lumens are readily usable according to the present invention. Additionally, in certain embodiments, the exterior port 18 may be selectively attachable to the body portion 12 such that the exterior port 18 may be fitted and removed by a physician. In those embodiments, the exterior port 18 may be threaded or snapped into the body portion 12, and any number of alternative mechanical means may be used for selectively attaching the respective components.

In the embodiment shown in FIGS. 1 through 7, a first skirt 20 and a second skirt 22 are disposed on the first surface 14 of the body portion 12 and bound the exterior port 18. Both skirts 20, 22 preferably comprise a durable tissue ingrowth material. Each of the first skirt 20 and the second skirt 22 preferably define an interior space adapted for receiving and surrounding the exterior port 18. More preferably, the second skirt 22 is arranged within the first skirt 20 and in closer proximity to the exterior port 18.

Accordingly, in this embodiment, the second skirt 22 defines a shape having a center that accommodates the exterior port 18 and a periphery that accommodates the center of the first skirt 20. As shown in the Figures, the first skirt 20 and the second skirt 22 are preferably annular or disk-like elements that are arranged concentrically about the exterior port 18. While this particular configuration is shown, design and engineering decisions might dictate alternative preferred shapes of the respective elements to suit particular purposes.

In this embodiment, the body portion 12 is further adapted to receive an interior port 24 that defines an interior lumen 32 for transporting fluids through the interior port 24. The interior port 24 is preferably cylindrical in shape, and the interior lumen 32 preferably defines a cylindrical surface through the interior lumen 32 of the interior port 24. Although a single lumen-type interior lumen 32 is illustrated throughout FIGS. 1 through 7, alternative designs including multiple lumens within the interior port 24 are compatible with the present invention. Additionally, in other embodiments, the device 10 may comprise no separate exterior port 18 and/or interior port 24.

Returning now to the embodiment shown in FIGS. 1 through 7, the interior port 24 may be selectively attachable to the body portion 12 such that a physician selectively may insert and remove the interior port 24. In embodiments having an interior port, the interior port 24 preferably defines a tip 26 or barb that is suitable for receiving a catheterization tube for delivering fluids into and out of the patient's system. In embodiments in which the interior port is selectively attachable, the interior port 24 may be threaded onto or snapped into the body portion 12, and/or joined by any alternative mechanical means consistent with the state of the art for selectively attaching the respective components.

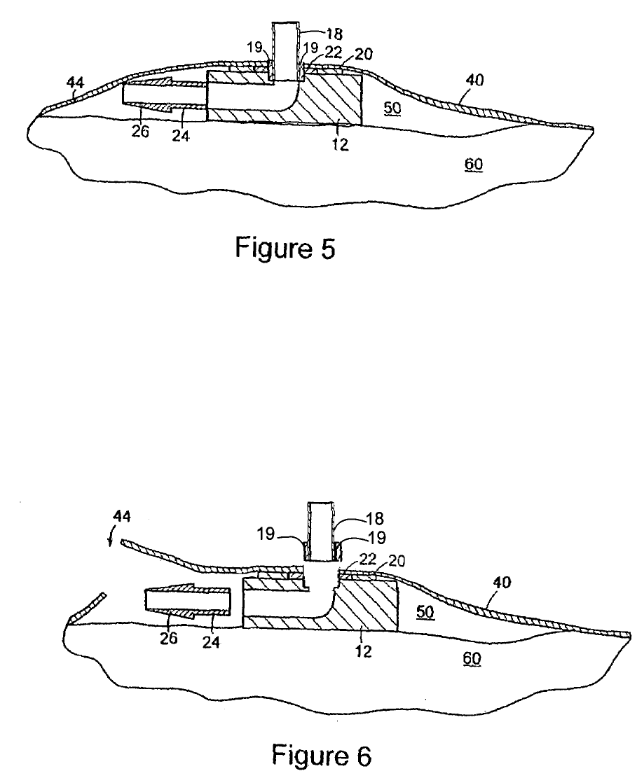

In use, the device 10 is disposed inside a patient underneath a living membrane, such as skin 40, and above other tissue 60, as shown in the embodiments of FIGS. 5 through 7. Generally, a physician will place the body portion 12 of the device under the skin 40 through an incision 44 made with a scalpel or other surgical instrument. Blunt dissection of the skin 40 from the tissue 60 defines a subcutaneous pocket 50 for receiving the body portion under the living membrane. Using a coring scalpel or other surgical instrument, a physician creates a port or opening in the skin 40 for receiving a conduit and/or the exterior port 18.

FIG. 5 shows an embodiment of the device 10 when newly installed within a patient such that both the first skirt 20 and the second skirt 22 contact both the body portion 12 and the patient's skin 40. This embodiment also depicts a tissue ingrowth ring 19 disposed about the exterior port 18 for interfacing with the patient's skin 40. The degree of attachment between the first skirt 20 and the device 10 is significantly less than the degree of attachment between the second skirt 22 and the device 10. As the healing process occurs over time, both the first skirt 20, the second skirt 22 and, in applicable embodiments, the tissue ingrowth ring 19 will become increasingly inter-connected to the patient's skin 40.

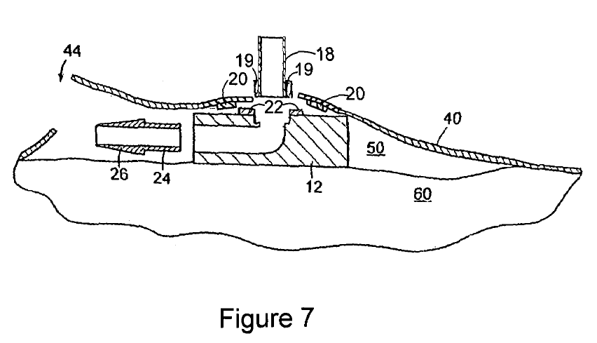

As the first skirt 20 and second skirt 22 become fully integrated with the patient's skin 40, the device 10 becomes more firmly secured within the patient. If after a short period of time the device 10 requires removal from the patient's body through normal surgical means, both the first skirt 20 and the second skirt 22 allow for complete removal along with the device 10 with application of only a low or moderate level of force. If the device 10 requires removal following a longer period of implantation, during which time extensive tissue ingrowth has occurred, the device 10 of the present invention still enables non-traumatic removal through application of a low or moderate level of force because the first skirt 20 will detach from the device 10 while remaining attached to the patient's skin 40. FIGS. 6 and 7, show stages of removal of the device 10 after a period of implantation that has allowed for tissue ingrowth between the first skirt 20 and the skin 40. The first skirt 20 is selectively attached to the device 10 so as to detach from the device 10 under application of low or moderate force while remaining attached to the skin 40. Only the second skirt 22, which has a significantly smaller surface area than the first skirt 20, requires detachment from the skin 40, thereby allowing removal of the device 10 without causing trauma.

In one embodiment, a process for assembling the device 10 of the present invention includes selectively attaching the first skirt 20 and the second skirt 22 to the body portion 12 by mechanical or other means, including adhesives. One skilled in the art will know to select adhesive that is compatible with the material and/or materials comprising the first skirt 20 and the second skirt 22, which may be, for example, bioabsorbable material, Dacron®, or titanium mesh. Weakly bonding the first skirt 20 to the body portion 12 ensures that less force is required to detach the first skirt 20 from the body portion 12 than to detach the first skirt 20 from the skin 40.

In another embodiment, the first skirt 20 may bond only to the second skirt 22 which bonds to the body portion 12. For example, this may be achieved by manufacturing the first skirt 20 and second skirt 22 from a single piece of material having a frangible line interposed between the two skirts 20, 22 and by applying adhesive only between the second skirt 22 and the body portion 12 during assembly of the device 10. The bond between the first skirt 20 and second skirt 22 thus will be weaker than the ingrowth bond between the first skirt 20 and the patient's skin 40 such that removal of the device 10 and the second skirt 22 adhered thereto results in a failure of the bond between the skirts 20, 22 at the frangible line therebetween. This failure along the frangible line leaves the first skirt 20 behind and attached to the patient's skin 40.

In yet another embodiment, the first skirt 20 is made from a material that has an inherently weaker internal structure as compared to the material of the second skirt 22. Both the first skirt 20 and second skirt 22 may be securely bonded to the body portion 10 by mechanical means, including adhesives. When a physician removes the device 10 from a patient, the weaker material of the first skirt 20 fails within itself, breaking apart in a plane substantially parallel to the skin 40. This failure mechanism leaves a lower portion of the first skirt 20 attached to the body portion 12 and an upper portion portions of the first skirt 20 attached to the skin 40.

In a similar embodiment, both the first skirt 20 and second skirt 22 may be manufactured from this weaker material, thus eliminating a requirement for a separate concentric section of non-detachable material.

In yet another embodiment, the first skirt 20 and/or second skirt 22 may attach to the body portion 12 via a bioabsorbable adhesive. The adhesive provides a secure initial bond, but over time, that attachment gradually weakens. The first skirt 20 and/or second skirt 22 initially adhered to the body portion 12 via the bioabsorbable adhesive separate easily from the body portion 12 after a period of time during which the bioabsorbable adhesive has degraded. Removal of the body portion 12 thereby leaves behind the first skirt 20 and/or second skirt 22 attached to the skin 40:

Similarly, in all embodiments, the tissue ingrowth material and/or materials comprising the first skirt 20, second skirt 22, and, in relevant embodiments, the tissue ingrowth ring 19, may comprise a varied scaffold matrix comprising an anisotropic structure having areas of varied density. Accordingly, select, less dense scaffold regions may enable a living membrane, such as skin 40, to more firmly attach to the ingrowth material of the skirts 20, 22 in those regions than in more dense surrounding regions. Upon removal of the device 10, the firmly ingrown region of the skirts 20, 22 will break away from the body portion 12 at the interfaces between the less dense and more dense regions, and the surrounding regions having less tissue ingrowth or no tissue ingrowth will remain attached to the body portion 12.

In all embodiments, the material comprising the skirts 20, 22 may be bioabsorbable such that the portions remaining attached to the patient's skin 40 degrade and are fully absorbed over time by the patient's body. In yet another embodiment, the detachable first skirt 20 and/or detachable second skirt 22 may adhere to a body portion 12 that has at least one bioabsorbable section. In this embodiment, the first skirt 20, and/or the second skirt 22, and at least one bioabsorbable section of the body portion 12 initially establish tissue ingrowth with a living membrane. Upon removal, the first skirt 20 and/or the second skirt 22, and the at least one bioabsorbable section of the body portion 12 all separate from the removed portion of the device 10 and remain behind safely within the patient. In yet another embodiment, the entire body portion 12 may be manufactured from a bioabsorbable material, enabling that entire base portion to remain behind. This embodiment eliminates a need for having to reopen an insertion pocket for removal of some of the device 10 or the entire device 10.

For embodiments in which the first skirt 20 and/or second skirt 22 and/or body portion comprise bioabsorbable tissue ingrowth material, the device 10 remains attached securely within the patient's body for a known duration of time, and the bioabsorbable material gradually mechanically degrades at an expected point in time. A host of bio-absorbable materials are known in the art of medical devices. Preferable materials for the present invention include polymers such as polyglycolide, polylactide, l-lactide, poly(dl-lactide), polycolactide, poly(ε-caprolactone), polydiaxanone, polyglyconate or poly(lactide-co-glycolide) (DLPLG). The present invention may incorporate different formulas of DLPLG including 85/15, 75/25, 65/35 and 50/50 wherein the various ratios are indicative of the mixtures of glycolide and dl-lactide, respectively. The higher the proportional ratio of glycolide to dl-lactide, the greater the period of degradation of the DLPLG polymer.

Each of these polymers has unique mechanical and thermal properties, as well as variable degradation intervals. Accordingly, the type of polymer selected for the first skirt 20 and/or second skirt 22 is at least partially dependent upon the mechanical requirements of the application as well as the time period during which the device 10 will remain placed within a patient. For example, polyglycolide will remain in the human body for approximately six to twelve months, while polylactide will remain for more than twenty-four months. Similarly, poly(dl-lactide) will remain intact within the body for approximately twelve to sixteen months, while polycolactide will last over two years in a patient's body. Poly(ε-caprolactone), polydiaxanone, polyglyconate and the various forms of poly(lactide-co-glycolide) all have a duration of between one and twelve months. Thus, the type of material selected should be selected at least in part for its durability and interval of degradation. Longer-term implantations would require materials with a longer half-life, while the opposite is true for shorter-term applications of the device 10.

In certain embodiments, the first skirt 20 and/or second skirt 22 are preferably comprised of a non-bio-absorbable material, such as for example polyester fabric, woven polyurethane or titanium mesh. Similarly, the body portion 12 and the respective ports are preferably comprised of durable, non-bio-absorbable materials such as polyurethane, polysulfone, polycarbonate, silicone, titanium or stainless steel. Any combination of materials is also suitable for the foregoing components, such that they will be biocompatible, lightweight, durable and easy to assemble and maintain once inside the patient's body.

Although the second skirt 22, the body portion 12 and, in applicable embodiments, the respective ports 18, 24 are preferably composed of those materials described above, in some cases manufacturing these components from bioabsorbable materials may be preferable. As varying rates of degradation correspond to different types of bioabsorbable materials, as noted above, some embodiments of the device 10 may incorporate materials that will have degradation periods on the order of years as opposed to months. Thus any component of the present device may be composed wholly or partially of bioabsorbable materials selected according to the particular application and the interval during which the device 10 will function within a patient. For example, the first skirt 20 may be composed of polyglycolide, while the remaining components may be composed of polycolactide, which will render the remaining components effectively permanent relative to the first skirt 20 over the expected duration of implantation. Additionally, some or all of the body portion 12 also may be constructed from materials having known rates of bioabsorbability. Those bioabsorbable portions of the body portion 12 may remain behind in the patient in order facilitate removal of the non-bioabsorbable portions of the device 10, and such that the bioabsorbable portions of the body portion 12 may remain behind as a permanent, absorbable implant. Accordingly, the present invention can be constructed in a number of fashions depending upon the intended use of the device 10.

As described herein, the present invention includes a device 10 that is readily usable as a tissue ingrowth cuff or similar device for permitting vascular access to a patient for various kinds of catheterization or dialysis procedures. Although described herein with particular application to catheterization, the present invention also is suited for use with non-vascular clinical uses, such as but not limited to peritoneal dialysis and cardiac rhythm management. The present invention may be used in any application requiring anchoring within tissue, sealing the point of insertion, and allowing for non-traumatic removal. Other non vascular clinical applications of the present invention may include, for example, anchoring a conduit or other device within organ tissue. For example, such applications might include anchoring a supra-pubic catheter used for bladder drainage or anchoring conductive elements, such as the power leads associated with many types of cardiac pacing and left ventricular heart assist devices (LVADS). All of these types of devices, which require eventual removal, need to traverse a living membrane, such as skin or other organ interfaces, in order to establish remote access for a certain period of implantation while preferably providing stable anchoring and a reduced risk of infection.

The device 10 of the present invention includes an at least partially detachable skirt 20, 22 disposed thereon, thus enabling uncomplicated, non-traumatic removal of the device 10 at the end of the therapeutic period. The force at which the detachable portion of the skirt 20, 22 will separate from the rest of the device 10 maybe pre-determined by varying the type or amount of adhesive used or by any other well-known means such as using a detachable material having an inherently lower strength modulus and higher friability than the non-detachable portion of the skirt.

Additionally, one skilled in the art will recognize that all embodiments of the ingrowth cuff of the present invention are treatable with bioactive and/or pharmaceutical substances that selective promote or retard cellular and tissue growth, regardless of material selection and/or overall geometry of the body portion 12.

Although the present invention has been described above with reference to preferred embodiments, materials, and clinical uses, one skilled in the art will understand that the scope thereof is not so limited. Those skilled in the art can devise numerous adaptations to the preceding description without departing from the spirit and scope of the present invention as defined in the following claims.

I claim:

1) An implantable medical device comprising:
a) a body portion sized and shaped for placement within a patient's physiology, the body portion comprising an internal lumen capable of receiving and anchoring a treatment component; and
b) a tissue ingrowth cuff material releasably attached to a surface of the body portion and adapted for ingrowth with a living membrane.

2) The implantable medical device of claim 1 wherein the treatment component comprises a fluid conduit having one or more lumens.

3) The implantable medical device of claim 1 wherein the treatment component comprises one or more conductive elements.

4) The implantable medical device of claim 1 wherein the treatment component comprises one or more fiber optic cables.

5) The implantable medical device of claim 1 wherein the treatment component comprises an exterior port.

6) The implantable medical device of claim 5 further comprising an interior port defining an interior lumen, the interior port selectively joinable to the body portion such that the interior port and interior lumen are substantially orthogonal to the exterior port.

7) The implantable medical device of claim 1 wherein the body portion is sized and shaped for subcutaneous placement.

8) The implantable medical device of claim 1 wherein the tissue ingrowth cuff material comprises a first skirt and a second skirt.

9) The implantable medical device of claim 8 wherein the first and second skirts are concentric

10) The implantable medical device of claim 8 wherein the first skirt is selectively attachable to the body portion.

11) The implantable medical device of claim 8 wherein the first skirt and second skirt are affixed to the body portion with one or more adhesives.

12) The implantable medical device of claim 11 wherein the adhesive affixing the first skirt to the body portion forms a weaker bond than the adhesive affixing the second skirt to the body portion.

13) The implantable medical device of claim 11 wherein the one or more adhesives are bioabsorbable.

14) The implantable medical device of claim 8 wherein the first and second skirt are formed of a single sheet of material having a frangible line interposed between the first and second skirt.

15) The implantable medical device of claim 14 wherein only the second skirt is affixed to the body portion such that only the second skirt remains affixed to the body portion upon removal and such that the first skirt separates from the second skirt and remains attached to the living membrane.

16) The implantable medical device of claim 8 wherein the first skirt is comprised of a tissue ingrowth material having an internal structure that is inherently weaker than the bonds between the cuff and the body portion and between the cuff and ingrown tissue such that upon removal of the device the tissue ingrowth material fails within itself in a plane substantially parallel to the living membrane.

17) The implantable medical device of claim 16 wherein the weaker ingrowth material of the first skirt is bioabsorbable.

18) The implantable medical device of claim 8 wherein the first and/or second skirt are formed from a material having an anisotropic structure comprising regions of varied tissue ingrowth capability such that some regions are capable of stronger tissue ingrowth than other regions.

19) The implantable medical device of claim 8 wherein the first skirt comprises bio-absorbable tissue ingrowth material.

20) The implantable medical device of claim 17 wherein the bio-absorbable material is selected from a group consisting of polyester fabric, woven polyurethane, titanium mesh, polyglycolide, polylactide, I-lactide, poly(dl-lactide), polycolactide, poly(ε-caprolactone), polydiaxanone poly(lactide-co-glycolide), and polyglyconate.

21) The implantable medical device of claim 1 wherein the tissue ingrowth material is selected from a group consisting of polyurethane, polysulfone, polycarbonate, silicone, titanium and stainless steel.

22) The implantable medical device of claim 1 wherein the body portion material is selected from a group consisting of polyurethane, polysulfone, polycarbonate, silicone, titanium and stainless steel.

23) The implantable medical device of claim 1 wherein at least a portion of the body portion is bioabsorbable.

24) The implantable medical device of claim 21 wherein the bio-absorbable material is selected from a group consisting of polyester fabric, woven polyurethane, titanium mesh, polyglycolide, polylactide, I-lactide, poly(dl-lactide), polycolactide, poly(ε-caprolactone), polydiaxanone poly(lactide-co-glycolide), and polyglyconate.
